(19) United States
(12) Patent Application Publication
(10) Pub. No.: US 2003/0082721 A1
(43) Pub. Date: May 1, 2003

(54) Transgenic animals expressing androgen receptor complex-associated protein

(76) Inventor: Tai-Jay Chang, Taipei (TW)

Correspondence Address: FISH & RICHARDSON PC, 225 FRANKLIN ST, BOSTON, MA, 02110, US

(21) Appl. No.: 10/206,566

(22) Filed: Jul. 25, 2002

Related U.S. Application Data
(63) Continuation-in-part of application No. 09/781,693, filed on Feb. 12, 2001.
(60) Provisional application No. 60/262,312, filed on Jan. 17, 2001.

(51) Int. Cl. A01K 67/027; C07H 21/04; C12P 21/02; C12N 5/06; C07K 14/72

(52) U.S. Cl. 435/69.1; 435/320.1; 435/325; 530/350; 536/23.5; 800/18

(57) ABSTRACT

Transgenic animals expressing an androgen receptor complex-associated protein gene. These animals serve as models for developing drugs to treat cancer, e.g., liver cancer.

### RELATED APPLICATIONS

This application is a continuation-in-part of and claims priority to U.S. application Ser. No. 09/781,693, filed Feb. 12, 2001 and U.S. Provisional Application Serial No. 60/262,312, filed Jan. 17, 2001, the contents of which are incorporated herein by reference.

### BACKGROUND

A variety of genes that are overexpressed in tumor cells relative to healthy cells have been identified. It is expected that the identification of such genes will provide drug targets for anti-cancer drug development and for cancer diagnostics. The number of steroid receptors (e.g., androgen receptors) in liver tumor cells appears to be increased relative to their adjacent healthy liver cells.

Steroid hormones generally exert their physiological effects by binding to their specific nuclear receptors to form complexes that in turn act as transcription factors. The complexes bind to specific nucleotide sequences (steroid responsive elements) in the promoters of steroid-responsive genes to facilitate transcription of those genes.

### SUMMARY

The invention is based on the discovery of a mouse gene encoding an androgen receptor complex-associated protein (ARCAP), which is 85% identical to human ARCAP. Human ARCAP has been found to be overexpressed in hepatoma cells (relative to adjacent normal cells), and binds to an androgen receptor and augments the ability of the receptor to transactivate an androgen-responsive gene. As a homolog to the human ARCAP gene, the mouse ARCAP gene can be used to generate a transgenic mouse that serves as an animal model for developing drugs to treat cancer (e.g., liver cancer).

The full-length mouse ARCAP cDNA (designated SEQ ID NO:1), with the start and stop codons underlined, is shown below:

(SEQ ID NO:1)
GAGAGTATGAGGCGAGCCGTGGCCCGGGTGCCTCTCCGCTCCCGGGGAGA
AGGCGCGGCCGTGGCTGCCGCCCTCTGAGTCGCGGCGCCGGCGAGGCCCC
GGGGCGCGCGCATGGTGCTGGTGCCGCTCGGGTGTTGATCGGCCTGTCCC
CTCCCTCTCTTCCCCTCCCCACCCCCCGCGGTGGTCTCCCCTTTCCCACC
CCAGCCCTTGCGGAGCCATGGCTCGGAGTGGCTCCTGCCCGCACCTGTTG
TGGGACGTGAGGAAAAGGTCCCTTGGGCTGGAGGACCCGTCCCGGCTGAG
GAGCCGCTACCTGGGAAGAAGAGAATTTATCCAAAGATTAAAACTTGAAG
CAACTTTAAATGTGCATGATGGCTGTGTTAATACAATCTGTTGGAATGAC
ACTGGAGAATATATTTTATCTGGCTCTGATGACACTAAACTTGTAATTAG
TAATCCATACAGCAGAAAGGTTTTGACAACCATCCGTTCAGGGCATCGAG
CAAATATATTTAGTGCAAAGTTTTTGCCGTGCACAGATGATAAGCAGATT
GTGTCTTGCTCTGGAGATGGAGTCATATTTTATACTAACATTGAGCAAGA
TGCAGAAACTAACAGACAGTGCCAATTTACGTGCCATTATGGAACTACTT
ATGAGATTATGACTGTACCAAACGACCCTTACACCTTTCTGTCCTGTGGT
GAAGATGGAACTGTTAGGTGGTTTGACACACGCATCAAAACCAGTTGCAC
AAAAGAAGACTGTAAAGATGATATTTTAATCAACTGTAGGCGTGCTGCCA
CATCTGTGGCTATTTGTCCCCCAGTACCATATTACCTTGCTGTGGGTTGT
TCTGACAGCTCAGTACGGATTTATGATCGGCGAATGCTGGGCACAAGAGC
TACAGGGAATTATGCAGGCCGAGGAACTACTGGAATGGTTGCTCGATTTA
TACCTTCTCATCTTAGTAACAAATCATGCAGAGTGACATCACTGTGTTAC
AGTGAAGATGGTCAAGAGATTCTTGTCAGTTATTCTTCAGACTACATCTA
TCTTTTTGACCCCAAAGATGATACTGCACGAGAACTTAAAACTCCTTCTG
CAGAGGAGAGGCGAGAAGAGTTACGACAGCCTCCAGTTAAGCGCTTGAGA
CTTCGTGGTGATTGGTCAGATACTGGTCCCAGAGCACGGCCAGAAAGTGA
ACGAGAACGAGATGGAGAGCAAAGTCCCAATGTGTCACTGATGCAGAGAA
TGTCTGATATGTTATCAAGGTGGTTTGAAGAAGCAAGTGAAGTTGCACAA
AGCAACAGAGGAAGAGGAAGACCTCGGCCCAGAGGTGGAACAAATCAGCC
AGATGTTTCAACTCTTCCTACGGTTCCATCAAGTCCTAATTTGGAAGTGT
GTGAAACTGCAATGGATGTAGACATGCCAGCTGCACTTCTTCAGCCTTCT
ACATCCTCTACAGATCCAGTTCAGGCTCAGGCAGCCACAGCCGCCATAGA
AAGCCCTCGTTCCAGCTCGTTGCTGTCTTGCCCAGACAGTGAACCGAGGC
AGTCTGTTGAGGCGTCTGGACACCATGCACATCATCAGTCAGATTCTCCT
TCTTCTGTGGTTAACAAACAGCTGGGATCCATGTCACTTGATGAGCAACA
GGATAACAGTAATGAGAGGCTGAGCCCCAAACCAGGGACAGGTGAACCCG
TTTTAAGTTTGCACTACAGCACAGAAGGAACAACTACAAGCACAATAAAA
CTGAACTTTACAGATGAATGGAGCAGTACAGCCTCAAGTTCCAGAGGAAA
TGGGAGCCATTGCAAATCTGAGGGTCAGGAAGAATGCTTGGTCCCTCCGA
GCTCTGTGCAGCCACCGGAAGGAGACAGTGAAACAAGAGCTCCTGAAGAA
CTATCAGAGAAAGGAACACTTCCAGAAAACCTCACTCAAAACCAGATAGA
TACAGCACAACTTGATAACTTCCCAGCTGAGCCATTGGATTCTAACTCAG
GAGAGAAGAATAACCCAAGTCAGGACAGCCCTTGTGGGCTTCCAGAAGAA
GGCACTTTGTCTGAAACAGACAGGGAGACTTGTGAGCAGGCCAGCACTGA
GAGTGCTACCAGGCATGCTAGCACCAAGCCTGAACTCCCATCCCAGACAG
AAGCCATTGAGCAGGCCAGCACTGAGAGTGCTACCAGGCATACCAGTGCC
AATCCTGAACTCCCATCCCAGACAGAAGCCATAGCACCTTTAGCTCATGA
AGACCCATCTGCCAGGGACTCTGCTCTCCAGGACACAGATGACAGCGATG
ATGATCCGGTCTTGATCCCTGGTGCAAGATACCGAACAGGACCTGGTGAT
AGACGCTCCGCTGTTGCCCGCATTCAGGAGTTCTTCAGGAGGAGAAAAGA
AAGGAAAGAAATGGAAGAGCTGGATACTTTGAACATTAGGAGGCCACTAG
TAAAGATGGTTTATAAGGGCCACCGCAACTCCCGGACAATGATAAAAGAA
GCCAATTTCTGGGGTGCTAACTTTGTAATGAGCGGTTCCGATTGTGGCCA
TATCTTCATCTGGGACCGGCACACTGCGGAGCATTTGATGCTTCTGGAAG
CTGATAATCATGTGGTCAACTGCCTGCAGCCCCATCCGTTTGACCCAATT
CTAGCCTCATCTGGCATAGATTATGACATAAAGATCTGGTCGCCACTAGA
AGAGTCAAGAATTTTTAATCGAAAACTTGCTGATGAAGTTATAACTCGGA
ATGAACTCACGCTGGAAGAGACTCGGAACACCATCACCGTCCCAGCCTCT
TTCATGTTGAGGATGTTGGCGTCACTGAATCATATCCGAGCTGACCGTCT
GGAGGGTGACAGATCAGAAGGTTCAGGTCAGGAGAATGAAAATGAGGATG
AAGAATAAAGAACTCCTTGGCAAGCACTTAGATGTTCTGAGATTTGTATA
CGACATTTATTATATTTTTTTTCTTTACAGAACTTTAGTGCAATTTAAGG
CTATGGGTTTTTTTTTTTCTTTTTTTTTGGAGTTCTTCCCTATTTTGGGG
ATAACCAAACATTGGTTTGGAATGAGTGTGTGCATGAGTTGGGAGAGTGT
GTAAAACAAAGTAAGCAAAATGTTTTTTGAAACCTTTTGCCGTGTATGGA
GTCCCAAAAAAAAAAAAAAAAAAAAAAAAAAGCAAAGTGCAATACTTCCT
GACCCTCCGCTGTGGGAGCTTGGATCAATGCTGAAGTCATTTTCATTGTA
GTGAAAACGTTGGTTCAAATAAATTTCTACACTTGCCATTTGCAAAAAAA
AAAAAAAAAAAAAAAAAAAAAAGCGGCCGCTGAATTCTAG

The nucleotide sequence encoding the mouse ARCAP protein (i.e., from the ATG start codon to the codon immediately before the stop codon in SEQ ID NO:1) is designated SEQ ID NO:2. The mouse ARCAP protein (designated SEQ ID NO:3) encoded by the above cDNA is shown below:

(SEQ ID NO:3)
Met ala arg ser gly ser cys pro his leu leu trp
asp val arg lys arg ser leu gly leu glu asp pro
ser arg leu arg ser arg tyr leu gly arg arg glu
phe ile gln arg leu lys leu glu ala thr leu asn
val his asp gly cys val asn thr ile cys trp asn
asp thr gly glu tyr ile leu ser gly ser asp asp
thr lys leu val ile ser asn pro tyr ser arg lys
val leu thr thr ile arg ser gly his arg ala asn
ile phe ser ala lys phe leu pro cys thr asp asp
lys gln ile val ser cys ser gly asp gly val ile
phe tyr thr asn ile glu gln asp ala glu thr asn
arg gln cys gln phe thr cys his tyr gly thr thr
tyr glu ile met thr val pro asn asp pro tyr thr
phe leu ser cys gly glu asp gly thr val arg trp
phe asp thr arg ile lys thr ser cys thr lys glu
asp cys lys asp asp ile leu ile asn cys arg arg
ala ala thr ser val ala ile cys pro pro val pro
tyr tyr leu ala val gly cys ser asp ser ser val
arg ile tyr asp arg arg met leu gly thr arg ala
thr gly asn tyr ala gly arg gly thr thr gly met
val ala arg phe ile pro ser his leu ser asn lys
ser cys arg val thr ser leu cys tyr ser glu asp
gly gln glu ile leu val ser tyr ser ser asp tyr
ile tyr leu phe asp pro lys asp asp thr ala arg
glu leu lys thr pro ser ala glu glu arg arg glu
glu leu arg gln pro pro val lys arg leu arg leu
arg gly asp trp ser asp thr gly pro arg ala arg
pro glu ser glu arg glu arg asp gly glu gln ser
pro asn val ser leu met gln arg met ser asp met
leu ser arg trp phe glu glu ala ser glu val ala
gln ser asn arg gly arg gly arg pro arg pro arg
gly gly thr asn gln pro asp val ser thr leu pro
thr val pro ser ser pro asn leu glu val cys glu
thr ala met asp val asp met pro ala ala leu leu
gln pro ser thr ser ser thr asp pro val gln ala
gln ala ala thr ala ala ile glu ser pro arg ser
ser ser leu leu ser cys pro asp ser glu pro arg
gln ser val glu ala ser gly his his ala his his
gln ser asp ser pro ser ser val val asn lys gln
leu gly ser met ser leu asp glu gln gln asp asn
ser asn glu arg leu ser pro lys pro gly thr gly
glu pro val leu ser leu his tyr ser thr glu gly
thr thr thr ser thr ile lys leu asn phe thr asp
glu trp ser ser thr ala ser ser ser arg gly asn
gly ser his cys lys ser glu gly gln glu glu cys
leu val pro pro ser ser val gln pro pro glu gly
asp ser glu thr arg ala pro glu glu leu ser glu
lys gly thr leu pro glu asn leu thr gln asn gln
ile asp thr ala gln leu asp asn phe pro ala glu
pro leu asp ser asn ser gly glu lys asn asn pro
ser gln asp ser pro cys gly leu pro glu glu gly
thr leu ser glu thr asp arg glu thr cys glu gln
ala ser thr glu ser ala thr arg his ala ser thr
lys pro glu leu pro ser gln thr glu ala ile glu
gln ala ser thr glu ser ala thr arg his thr ser
ala asn pro glu leu pro ser gln thr glu ala ile
ala pro leu ala his glu asp pro ser ala arg asp
ser ala leu gln asp thr asp asp ser asp asp asp
pro val leu ile pro gly ala arg tyr arg thr gly
pro gly asp arg arg ser ala val ala arg ile gln
glu phe phe arg arg arg lys glu arg lys glu met
glu glu leu asp thr leu asn ile arg arg pro leu
val lys met val tyr lys gly his arg asn ser arg
thr met ile lys glu ala asn phe trp gly ala asn
phe val met ser gly ser asp cys gly his ile phe
ile trp asp arg his thr ala glu his leu met leu
leu glu ala asp asn his val val asn cys leu gln
pro his pro phe asp pro ile leu ala ser ser gly
ile asp tyr asp ile lys ile trp ser pro leu glu
glu ser arg ile phe asn arg lys leu ala asp glu
val ile thr arg asn glu leu thr leu glu glu thr
arg asn thr ile thr val pro ala ser phe met leu
arg met leu ala ser leu asn his ile arg ala asp
arg leu glu gly asp arg ser glu gly ser gly gln
glu asn glu asn glu asp glu glu

Accordingly, the invention features a substantially pure polypeptide or protein including an amino acid sequence at least 70% (e.g., at least 75, 80, 85, 90, 95, 98, or 100%) identical to SEQ ID NO:3. If the polypeptide includes a sequence that is 100% identical to SEQ ID NO:3, the polypeptide can contain up to 30 conservative amino acid substitutions. The invention also includes a substantially pure polypeptide encoded by a nucleic acid that hybridizes under stringent conditions to a probe the sequence of which is SEQ ID NO:2. The polypeptide binds to an androgen receptor and increases the ability of the androgen receptor to transactivate an androgen-responsive gene. These polypeptides can be used for producing ARCAP antibodies (either monoclonal or polyclonal). These antibodies in turn are useful for detecting the presence and distribution of ARCAP in tissues and in cellular compartments. For example, such antibodies can be used to diagnose cancerous liver tissue by determining whether ARCAP protein is expressed or overexpressed in the tissue. They can also be used to treat cancer (e.g., liver cancer) as described below.

The invention further features an isolated nucleic acid encoding a polypeptide of the invention, a vector including a nucleic acid of the invention, and a cell (in an animal model or in a culture) containing a nucleic acid of the invention. An example of a nucleic acid within the invention includes an isolated nucleic acid having a strand that hybridizes under stringent conditions to a single stranded probe, the sequence of which is SEQ ID NO:2 or the complement of SEQ ID NO:2. Such a nucleic acid can be at least 15 (e.g., at least 30, 50, 100, 200, 500, or 1000) nucleotides in length. These nucleic acids, vectors, and cells can be used for generating an animal model, diagnosing cancer (e.g., liver cancer), or producing the polypeptides of the invention. For instance, the nucleic acids of the invention can be used to diagnose liver cancer by determining whether ARCAP mRNA is being expressed or overexpressed in a tissue or cell. The nucleic acids can be used as primers in PCR-based detection methods, or as labeled probes in nucleic acid blots (e.g., Northern blots).

The invention also features a transgenic animal (e.g., a rodent such as a mouse) whose genome includes a transgene containing a nucleic acid of this invention and exhibits an increased androgen response as compared to a wild-type animal. The transgenic animal can be generated by introducing a nucleic acid of the invention into a cell such that a transcript is produced from the nucleic acid and the transcript is translated into a protein. These transgenic animals can be used as models for developing drugs to treat cancer (e.g., liver cancer).

In addition, the invention features a method of producing a polypeptide of the invention by culturing a cell described above, expressing the polypeptide in the cell, and isolating the polypeptide from the culture.

The invention further features a method of determining whether an animal sample (e.g., a blood sample) contains cancerous cells. The method involves providing a sample from an animal (e.g., a rodent such as a mouse), and determining the expression level of the ARCAP gene in the sample. If the expression level of the ARCAP gene in the sample is higher than that in a normal sample, it indicates that the animal sample contains cancerous cells (e.g., liver tumor cells). This method can be used for diagnosing cancer or monitoring treatment of cancer in an animal model.

Also within the scope of the invention is a method of treating cancer (e.g., liver cancer) in an animal (e.g., a rodent such as a mouse). The method involves identifying an animal that has a cancer cell expressing an ARCAP gene and treating the animal with a composition that blocks binding of ARCAP to an androgen receptor or decreases the ability of the androgen receptor to transactivate an androgen-responsive gene. The composition can contain an antibody of the invention or an anti-sense nucleic acid of the invention. This method can be used for screening drugs (including those used for gene therapy) and testing their efficacy in an animal model.

The term “substantially pure” as used herein in reference to a given polypeptide means that the polypeptide is substantially free from other biological macromolecules. The substantially pure polypeptide is at least 75% (e.g., at least 80, 85, 95, or 99%) pure by dry weight. Purity can be measured by any appropriate standard method, for example, by column chromatography, polyacrylamide gel electrophoresis, or HPLC analysis.

A “conservative amino acid substitution” is one in which an amino acid residue is replaced with another residue having a chemically similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

By hybridization under “stringent conditions” is meant hybridization at 65° C., 0.5×SSC, followed by washing at 45° C., 0.1×SSC.

The “percent identity” of two amino acid sequences or of two nucleic acids is determined using the algorithm of Karlin and Altschul (Proc. Natl. Acad. Sci. USA 87:2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. Where gaps exist between two sequences, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are used. See www.ncbi.nlm.nih.gov.

An “isolated nucleic acid” is a nucleic acid the structure of which is not identical to that of any naturally occurring nucleic acid or to that of any fragment of a naturally occurring genomic nucleic acid spanning more than three separate genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of different (i) DNA molecules, (ii) transfected cells, or (iii) cell clones, e.g., as these occur in a DNA library such as a cDNA or genomic DNA library.

Other features or advantages of the present invention will be apparent from the following detailed description, and also from the claims.

### DETAILED DESCRIPTION

The invention relates to transgenic non-human animals that express the ARCAP gene and methods of using the animals for the development of drugs for treatment or prevention of liver cancer.

As used herein, “transgenic non-human animal” includes the founder transgenic non-human animals and progeny of the founders, as well as cells and tissues from such animals. Transgenic non-human animals can be farm animals such as pigs, goats, sheep, cows, horses, and rabbits, rodents such as rats, guinea pigs, and mice, and non-human primates such as baboons, monkeys, and chimpanzees. Transgenic pigs and mice are particularly useful.

A transgenic non-human animal of the invention contains a nucleic acid encoding an ARCAP protein integrated within its genome. As used herein, the term “ARCAP protein” refers to a wild-type ARCAP protein or a functionally equivalent variant, e.g., a fragment of the full-length protein.

It is contemplated that it may be useful to identify intron sequences, if any, in a genome and include them in a nucleic acid encoding an ARCAP protein.

In transgenic non-human animals of the invention, the nucleic acid is operably linked to a regulatory element that can promote expression of the ARCAP gene, e.g., in liver. As used herein, the term “operably linked” refers to the placement of the regulatory element and nucleic acid in such a manner that the nucleic acid is transcribed. The regulatory element can be a liver-specific promoter, such as a PEPCK and albumin promoter.

The invention also features expression vectors suitable for generating transgenic non-human mediating expression in liver operably linked to a nucleic acid encoding an ARCAP protein as described above.

Various techniques known in the art can be used to introduce expression vectors into non-human animals to produce the founder lines of the transgenic non-human animals. Such techniques include, but are not limited to, pronuclear microinjection (U.S. Pat. No. 4,873,191), retrovirus mediated gene transfer into germ lines (Van der Putten et al., Proc. Natl. Acad. Sci. USA, 82:6148, 1985), gene targeting into embryonic stem cells (Thompson et al., Cell, 56:313, 1989), electroporation of embryos (Lo, Mol. Cell. Biol., 3:1803, 1983), and transformation of somatic cells in vitro followed by nuclear transplantation (Wilmut et al., Nature, 385(6619):810-813, 1997; and Wakayama et al., Nature, 394:369-374, 1998). In one example, the expression vector is microinjected into an ovum or embryo of a non-human animal or into embryonic stem cells of a non-human animal.

Once transgenic non-human animals have been generated (e.g., a transgenic mouse generated according to the Current Protocol (Wiley, USA) and Manuplate the Mouse Embryo (Hogan Beddington and Costantini Lacy, CSHL Press), expression of the ARCAP gene can be assessed using standard techniques. Initial screening can be accomplished by Southern blot analysis or PCR techniques to determine whether or not integration of the transgene has taken place. See, for example, sections 9.37-9.52 of Sambrook et al., 1989, “Molecular Cloning, A Laboratory Manual”, second edition, Cold Spring Harbor Press, Plainview; N.Y., for a description of Southern analysis. Expression of the nucleic acid encoding an ARCAP protein in the tissues of the transgenic non-human animals can be assessed using techniques that include, but are not limited to, Northern blot analysis of tissue samples obtained from the animal, in situ hybridization analysis, and reverse-transcriptase PCR (RT-PCR).

Transgenic non-human animals of the invention can be used as models for liver cancer. In particular, these animals can be used to identify a compound or composition effective for treatment or prevention of liver cancer. Compounds or compositions can be identified by administering a test compound or composition to a transgenic non-human animal of the invention or by contacting the test compound or composition with an organ, a tissue (e.g., liver) or cells (e.g., liver cells) derived from the transgenic non-human animal. Effects of the test compound or composition on liver cancer of the transgenic non-human animal, organ, tissues or cells are evaluated. For example, the size of the cancer from clinical pathology identification can be assessed in the transgenic non-human animals. Test compounds or compositions that palliate the cancer symptoms can be effective for treatment or prevention of liver cancer.

Test compounds can be formulated into pharmaceutical compositions by admixing the compounds with pharmaceutically acceptable non-toxic excipients or carriers and administered to transgenic non-human animals of the invention by any route of administration. For example, parenteral routes such as subcutaneous, intramuscular, intravascular, intradermal, intranasal, inhalation, intrathecal, or intraperitoneal administration, and enteral routes such as sublingual, oral, or rectal administration can be used.

Without further elaboration, it is believed that one skilled in the art can, based on the description herein, utilize the present invention to its fullest extent. All publications recited herein are hereby incorporated by reference in their entirety.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

What is claimed is:

1. A substantially pure polypeptide comprising an amino acid sequence at least 70% identical to SEQ ID NO:3, wherein the polypeptide binds to an androgen receptor and increases the ability of the androgen receptor to transactivate an androgen-responsive gene.

2. The polypeptide of claim 1, wherein the amino acid sequence is at least 80% identical to SEQ ID NO:3.

3. The polypeptide of claim 2, wherein the amino acid sequence is at least 90% identical to SEQ ID NO:3.

4. The polypeptide of claim 3, wherein the amino acid sequence is at least 95% identical to SEQ ID NO:3.

5. The polypeptide of claim 4, wherein the amino acid sequence is SEQ ID NO:3.

6. A substantially pure polypeptide comprising the amino acid sequence of SEQ ID NO:3, with up to 30 conservative amino acid substitutions, wherein the polypeptide binds to an androgen receptor and increases the ability of the androgen receptor to transactivate an androgen-responsive gene.

7. A substantially pure polypeptide encoded by a nucleic acid that hybridizes under stringent conditions to a probe, the sequence of which is SEQ ID NO:2, wherein the polypeptide binds to an androgen receptor and increases the ability of the androgen receptor to transactivate an androgen-responsive gene.

8. An isolated nucleic acid encoding a polypeptide of claim 1.

9. The nucleic acid of claim 8, wherein the amino acid sequence is SEQ ID NO:3.

10. An isolated nucleic acid encoding a polypeptide of claim 6.

11. An isolated nucleic acid comprising a strand that hybridizes under stringent conditions to a single stranded probe, the sequence of which is SEQ ID NO:2 or the complement of SEQ ID NO:2.

12. The nucleic acid of claim 11, wherein the nucleic acid encodes a polypeptide that binds to an androgen receptor and increases the ability of the androgen receptor to transactivate an androgen-responsive gene.

13. The nucleic acid of claim 12, wherein the amino acid sequence of the polypeptide includes SEQ ID NO:3.

14. The nucleic acid of claim 11, wherein the strand is at least 15 nucleotides in length.

15. A vector comprising a nucleic acid of claim 8.

16. The vector of claim 15, wherein the amino acid sequence is SEQ ID NO:3.

17. A vector comprising a nucleic acid of claim 10.

18. A vector comprising a nucleic acid of claim 11.

19. The vector of claim 18, wherein the nucleic acid encodes a polypeptide that binds to an androgen receptor and increases the ability of the androgen receptor to transactivate an androgen-responsive gene.

20. A cell comprising a nucleic acid of claim 8.

21. The cell of claim 20, wherein the amino acid sequence is SEQ ID NO:3.

22. A cell comprising a nucleic acid of claim 10.

23. A cell comprising a nucleic acid of claim 11.

24. The cell of claim 23, wherein the nucleic acid encodes a polypeptide that binds to an androgen receptor and increases the ability of the androgen receptor to transactivate an androgen-responsive gene.

25. A transgenic animal whose genome comprises a transgene containing a nucleic acid of claim 8, wherein the transgenic animal exhibits an increased androgen response as compared to a wild-type animal.

26. The transgenic animal of claim 25, wherein the animal is a rodent.

27. The transgenic animal of claim 26, wherein the animal is a mouse.

28. The transgenic animal of claim 25, wherein the amino acid sequence is SEQ ID NO:3.

29. The transgenic animal of claim 28, wherein the animal is a rodent.

30. The transgenic animal of claim 29, wherein the animal is a mouse.

31. A transgenic animal whose genome comprises a transgene containing a nucleic acid of claim 10, wherein the transgenic animal exhibits an increased androgen response as compared to a wild-type animal.

32. The transgenic animal of claim 31, wherein the animal is a rodent.

33. The transgenic animal of claim 32, wherein the animal is a mouse.

34. A transgenic animal whose genome comprises a transgene containing a nucleic acid of claim 11, wherein the transgenic animal exhibits an increased androgen response as compared to a wild-type animal.

35. The transgenic animal of claim 34, wherein the animal is a rodent.

36. The transgenic animal of claim 35, wherein the animal is a mouse.

37. A method of producing a polypeptide, the method comprising culturing a cell of claim 20, expressing the polypeptide in the cell, and isolating the polypeptide from the culture.

38. A method of producing a transcript, the method comprising introducing a nucleic acid of claim 11 into a cell, wherein a transcript is produced from the nucleic acid.

39. An isolated antibody that binds to a polypeptide comprising an amino acid sequence of SEQ ID NO:3.

40. A method of determining whether an animal sample contains cancerous cells, the method comprising:
providing a sample from an animal, and
determining an expression level of an androgen receptor complex-associated protein gene in the sample,
wherein the expression level of the androgen receptor complex-associated protein gene in the sample, if higher than that in a normal sample, indicates that the animal sample contains cancerous cells.

41. The method of claim 40, wherein the sample is a blood sample.

42. The method of claim 40, wherein the cancerous cells are liver tumor cells.

43. The method of claim 40, wherein the animal is a rodent.

44. The method of claim 43, wherein the animal is a mouse.

45. A method of treating cancer in an animal, the method comprising:
identifying an animal having a cancer cell that expresses an androgen receptor complex-associated protein gene, and
treating the animal with a composition that blocks binding of the androgen receptor complex-associated protein to an androgen receptor or decreases the ability of the androgen receptor to transactivate an androgen-responsive gene.

46. The method of claim 45, wherein the composition contains an antibody of claim 39.

47. The method of claim 45, wherein the composition contains a nucleic acid of claim 11.

48. The method of claim 45, wherein the cancer is a liver cancer.

49. The method of claim 45, wherein the animal is a rodent.

50. The method of claim 49, wherein the animal is a mouse.
